(19) United States
(12) Plant Patent Application Publication
(10) Pub. No.: US 2003/0188356 P1
(43) Pub. Date: Oct. 2, 2003

(54) Carpinus coreana plant named 'KURO BIJIN''

(76) Inventor: Hikaru Fujinami, Kawaguchi City (JP)

Correspondence Address: CHRISTIE, PARKER & HALE, LLP, 350 WEST COLORADO BOULEVARD, SUITE 500, PASADENA, CA, 91105, US

(21) Appl. No.: 10/107,905

(22) Filed: Mar. 26, 2002

(51) Int. Cl. A01H 5/00

(52) U.S. Cl. PLT/226

(57) ABSTRACT

A new plant variety of Carpinus coreana characterized by Chocolate Brown leaf color throughout the growing season.

### BACKGROUND OF THE INVENTION

The present invention relates to a new and distinct variety of Carpinus coreana Nak., which was discovered as a seedling selection in a cultivated planting of an unnamed, unpatented Carpinus coreana in Kawaguchi City, Saitama Prefecture, Japan by the originator, Mr. Hikaru Fujinami. The varietal denomination of the new variety is ‘KURO BIJIN’.

The genus Carpinus is included in the family Betulaceae which comprises about 6 genera and over 100 species of deciduous, mostly monoecious trees and shrubs, native mostly to the Northern Hemisphere. Carpinus comprises approximately 35 species of deciduous, monoecious, medium or small sized trees originating in the Northern Hemisphere, many of which possess desirable ornamental characteristics.

Carpinus coreana possesses upright growth habit, with pendulous branches, broadly ovate leaves up to 5 cm long, with lustrous green foliage. Veins in pairs 10 to 12, hairy beneath, not impressed above. It generally possesses ornamental characteristics useful in the landscape.

### SUMMARY OF THE INVENTION

The new variety was discovered as a seedling selection in a controlled planting of Carpinus coreana and differs from other cultivars known to the inventor in having Chocolate Brown leaf color throughout the growing season. Asexual reproduction of the new variety by grafting, performed in Kawaguchi City, Saitama Prefecture, Japan; Vacaville, Calif.; and Lewisberry, Pa. have confirmed that the distinctive characteristics of the new variety are stable and transmitted to succeeding generations, and the new variety reproduces true to type.

### COMPARISON WITH PARENT

‘KURO BIJIN’ is distinguished from its parent and all other varieties of Carpinus coreana of which I am aware by its Chocolate Brown leaf color throughout the growing season.

### BRIEF DESCRIPTION OF ILLUSTRATION

The accompanying photographic illustration shows a specimen of the new cultivar in a photo illustration as true to color as is reasonably possible to make in an illustration of this character.

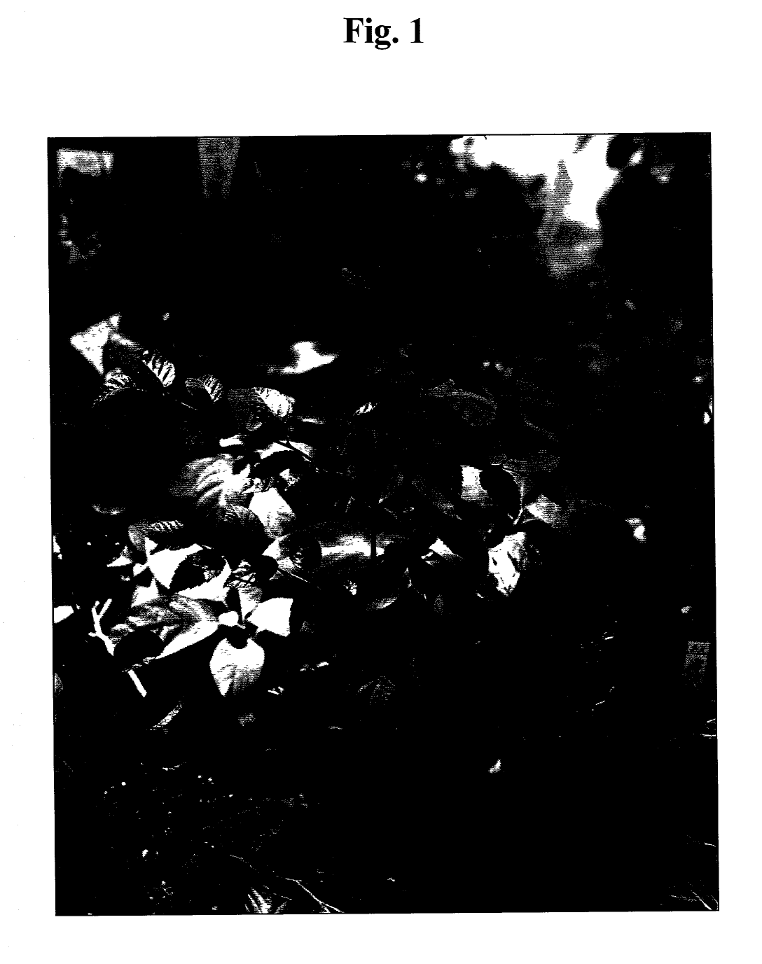

FIG. 1 illustrates the plant's unique foliage coloration.

### DESCRIPTION OF THE NEW VARIETY

‘KURO BIJIN’ has not been observed under all possible environmental, cultural and light conditions. The following observations and descriptions are of plants grown in Vacaville, Calif., Lewisberry, Pa., and Saitama Prefecture, Japan. In this description, color references are to The Royal Horticultural Society Colour Chart (2001) and terminology used in the color descriptions herein refers to plate numbers in this color chart. Phenotypic expression may vary with light intensity, cultural, and environmental conditions.

Classification:

Botanical.—Carpinus coreana Nak. ‘KURO BIJIN’.

Parentage.—Seedling selection from a controlled planting of Carpinus coreana (unnamed, unpatented).

Propagation.—Grafting.

Plant:

Size.—10 meters.

Size observed.—1 meter.

Habit.—Upright plant with arching branches and pendulous branch tips.

Branching.—Typical Quantity Branches: More than 1000, pendulous tips Observed Quantity Branches: 15 Branch Diameter: 1 cm at base Color: Near Brown Group 200A Leticiles: Near Grey-Brown Group 199B Color of new shoots: Near Brown Group N200A.

Leaf:

Shape.—Broadly ovate.

Length.—4 to 5 cm.

Width.—3 cm.

Arrangement.—Alternate.

Margin.—Double serrate.

Texture/Substance.—Lustrous throughout.

Color.—

The following color readings are of 2 year-old specimens grown in 5-gallon nursery containers in Vacaville, Calif. and Lewisberry, Pa. Young Foliage Upper side: Near Greyed-Purple Group N186 Underside: Near Greyed-Purple Group N186 Mature Foliage Upper side: Near Violet-Blue Group N92A Underside: Near Violet-Blue Group N92A Venation Pattern Upper Surface: Veins in 10 to 12 pairs, not impressed Under Surface: Fine hairs along mid rib

Petioles.—Size: 8 mm Color: Near Grey-Orange Group 176B Texture: Smooth.

Internodes.—1.5 to 3.0 cm.

Leaf buds.—Color: Near Brown Group 200B Size: Length: 5 mm Shape: Conical; slender.

Hardiness.—USDA Zone 7 (0° to 10° F.).

Vigor.—Moderate.

Pests/Diseases.—None noted.

Inflorescence: None yet observed

Reproductive Organs: None yet observed

I claim:

1. A new plant variety of Carpinus coreana of the variety substantially as shown and described.
